(19) United States
(12) Patent Application Publication
(10) Pub. No.: US 2010/0030201 A1
(43) Pub. Date: Feb. 4, 2010

(54) APPARATUS FOR SKIN TREATMENT

(75) Inventors: Ahmed Bitter, London (GB); Kevin Herbert, Gloucestershire (GB)

Correspondence Address: YOUNG & THOMPSON, 209 Madison Street, Suite 500, Alexandria, VA, 22314, US

(73) Assignee: THE DEZAC GROUP LIMITED, Gloucestershire (GB)

(21) Appl. No.: 12/519,019

(22) PCT Filed: Dec. 14, 2007
(86) PCT No.: PCT/GB07/04803

§ 371 Date: Jul. 14, 2009

(30) Foreign Application Priority Data
Dec. 16, 2006 (GB) 0625112.8

(51) Int. Cl. A61B 18/20; A61B 17/50

(52) U.S. Cl. 606/3; 606/7; 606/9; 606/133

(57) ABSTRACT

A portable hair treatment device 1 includes a hand held portion or wand 2 containing a laser and associated optics for effecting skin treatment. The device includes a main base unit 3 including a recess shaped to receive the hand held unit 2 when not in use. At least one of the base unit 3 and the wand 2 includes a portable power source, e.g. a battery, to enable self-powered use.

This invention relates to apparatus for skin treatment and in particular, but not exclusively, to the treatment of human or animal skin using laser radiation to effect a cosmetic and/or therapeutic treatment, for example hair removal.

It is already known to use laser treatment for hair removal. Laser radiation is directed towards the skin with the radiation being absorbed in the hair follicle and on the skin surface. The wavelength of the laser radiation is selected so as to be absorbed by melanin in the follicle so that the hair is heated to a temperature which causes it to stop growth. Although incidence of the laser radiation on the skin can also cause local skin heating, the heating of the hair follicle is much more acute.

Laser removal targeting one hair at a time with a narrowly focused laser beam is relatively inefficient and time consuming. To improve the time for treatment, many modern laser hair removal devices perform hair removal by focusing a number of lasers onto an area so as to treat a number of hair follicles simultaneously. However, the laser emitter is usually the component that consumes the most power in a laser hair remover device and so the provision of several laser emitters in a device results in a significant power demand. Also, it renders the device expensive to manufacture and therefore unsuitable for the general domestic market.

The relatively large electrical power demands of these prior art devices has resulted in them being physically bulky and dependent on a connection to mains electrical supply. Many end-users wish to treat body areas that are not easily reached, and in this context the aforementioned prior art devices are difficult to use and unsuitable for self-treatment.

The present invention, in at least some of its embodiments, addresses the above-mentioned problems and desires.

Accordingly, in one particular aspect this invention provides a portable hair treatment device for the treatment of the human or animal skin by laser radiation, the portable device including:

a laser radiation source for emitting a laser radiation beam; and

a portable power source providing an electrical output for powering the laser radiation source.

Preferably, the portable power source is a battery. Advantageously, the portable power source is a rechargeable battery, and the portable device further includes recharging means for enabling the rechargeable battery to be recharged. Alternative portable power sources, such as a fuel cell, might be envisaged.

The portable device may include more than one portable power source. Thus, the portable device may further include an ancillary portable power source for powering ancillary functions of the portable device.

In a preferred embodiment, the portable device includes a hand-held portion which houses the laser radiation source and a receiving unit for receiving the hand-held portion when it is not in use. The portable power source which provides the electrical output for powering the laser radiation source may be located in the receiving unit, or may be located in the hand-held portion. In embodiments in which this portable power source is located in the receiving unit, it is possible to provide an ancillary portable power source which is located in the hand-held portion. In embodiments in which the portable power source is located in the hand-held portion, it is possible for provide an ancillary portable power source located in the receiving unit.

Conveniently, the portable power source is a rechargeable battery, and the recharging means is configured so that the recharging commences when the receiving unit receives the hand-held portion.

In preferred embodiments, the portable device has a single laser radiation source. The use of a single radiation source rather than a plurality of laser radiation sources in combination with the provision of a portable power source enables the provision of a truly portable device which does not require a significant electrical supply.

The treatment effected may be exclusively cosmetic treatment, exclusively therapeutic treatment or a mixture thereof. For example, the treatment may comprise one or more of:

removal of hair

removal of tattoos or other skin pigmentation

treatment of blood vessels, for example visible capillaries such as port wine stains or surface veins, rosacea and similar discolorations

treatment to reduce the appearance of cellulite.

The laser radiation source may take many forms, but in one arrangement is a diode laser. The laser radiation source is preferably selected to emit radiation at a predetermined wavelength selected according to the nature of the treatment. In the case of laser treatment for hair removal the laser radiation source preferably has a wavelength of between 750 nm and 850 nm and more preferably about 808 nm. Again, the fluence of the laser radiation will be selected according to the nature of the particular treatment but for hair removal the fluence of the laser at the target site is preferably greater than 15 J/cm², and more preferably greater than about 20 J/cm².

In order to increase the operating life of the device, it is preferred that the radiation source emits a generally continuous beam once activated. However we do not exclude arrangements where the radiation source emits a pulsed beam.

Treatment using a small number of laser radiation sources, especially a single laser radiation source, is potentially difficult and time consuming because the area of the beam incident on the patient's skin is relatively small.

Accordingly, in a preferred embodiment, the portable device further includes movement means for moving said laser radiation beam in a predetermined pattern across the skin.

The movement means may comprise an optical element mounted for movement and drive means for moving said optical element to deflect the beam axis.

The optical element may take many forms such as a mirror or other reflector, but it is preferably in the form of a refracting means and, more specifically, a lens, mounted for movement, with there being drive means for moving the lens to deflect the beam axis. In one arrangement, the drive means is operable to shift the lens linearly in at least one direction generally transverse to the axis of the radiation beam emitted from the radiation source. More particularly, the drive means is preferably operable to shift said lens linearly in two generally orthogonal directions relative to the axis of the radiation beam. In this manner, taking a coordinate system in which the radiation beam is the Z axis, the beam may be deflected in the X and Y directions by corresponding shifting of the lens.

In another arrangement, the drive means may be operable to tilt the lens about at least one axis transverse to the beam axis and, more preferably, about two generally orthogonal axes.

The movement means may comprise said laser radiation source mounted for movement and drive means for moving said mounted laser radiation source to translate the beam axis. The drive means may be operable to shift the laser radiation source linearly in at least one direction generally transverse to the axis of the beam emitted from the laser radiation source. More particularly, the drive means is preferably operable to shift the laser radiation source linearly in two generally orthogonal directions relative to the axis of the beam.

In another arrangement, the drive means is operable to tilt the laser radiation source about at least one axis generally transverse to the axis of the beam emitted from the laser radiation source. More preferably, the drive means is operable to tilt the laser radiation source about two general orthogonal axes.

From the foregoing, it will be appreciated that it is possible to provide a laser radiation source relatively fixed with an optical element moving relative to the source, and also it is possible for the opposite arrangement to be employed whereby an optical element is kept relatively stationary whilst the laser radiation source is moved. Still further, it would be possible to effect relative movement by moving both the optical element and the laser radiation source, for example if there was a constraint on the movement of either due to the size of the device.

The drive means may take many different forms but is preferably electromagnetic comprising a permanent magnet and a coil with a current being passed through the coil to exert movement. However, other types of operation might be utilised, such as a piezo-electric device, an electric motor or a mechanical movement or a combination of these. In a mechanical arrangement the movement may be affected by providing a roller or other suitable element on the end of the device, which rotates as the device is drawn across the skin, the rotary motion of the roller being transmitted via a suitable transmission mechanism to effect said drive.

The device preferably includes control means operable to control the drive means to deflect said radiation beam. The control means may be operable to cause said beam to execute a scan pattern with a generally continuously moving scan spot. Alternatively, the control means may cause the beam to execute a scan pattern with discrete movements of a scan spot interspersed with dwell periods during which the spot is generally stationary. The extent of movement between dwell periods and/or the length of the dwell periods in a scan may be adjusted in accordance with the extent of deflection of the beam from its equilibrium position, so as to compensate for an increase or decrease in scan spot size as it moves.

In one arrangement, the control means is operable to dither the scan spot between selected regions so that selected regions receive multiple sequential exposures.

Preferably, the portable power source which provides the electrical output for powering the laser radiation source or an ancillary portable power source provides an electrical output for operating the movement means. The drive means and the control means can be powered in this way.

Whilst the invention has been described above it extends to any inventive combination as set out above or in the following description, drawings or claims.

The invention may be performed in various ways, and an embodiment thereof will now be described by way of example only, reference being made to the accompanying drawings, in which:

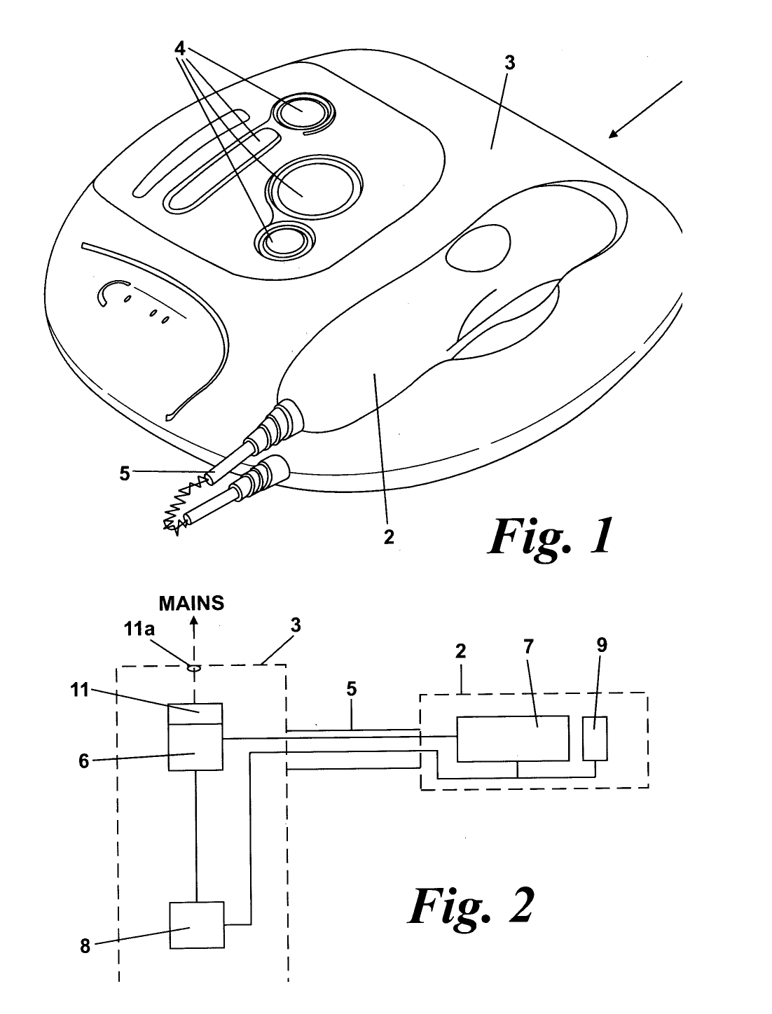

FIG. 1 is a perspective view of an embodiment of a device of the invention;

FIG. 2 is a schematic diagram showing the internal arrangement of the device of FIG. 1;

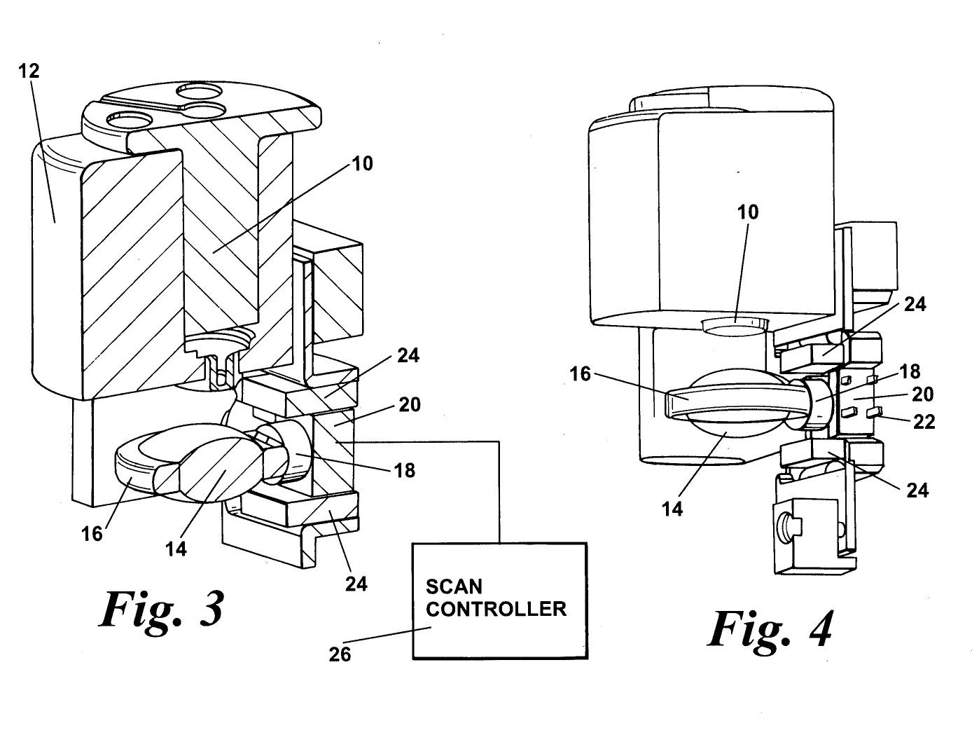

FIG. 3 is a perspective part cutaway view of a laser and deflector assembly for a hair treatment device in accordance with this invention;

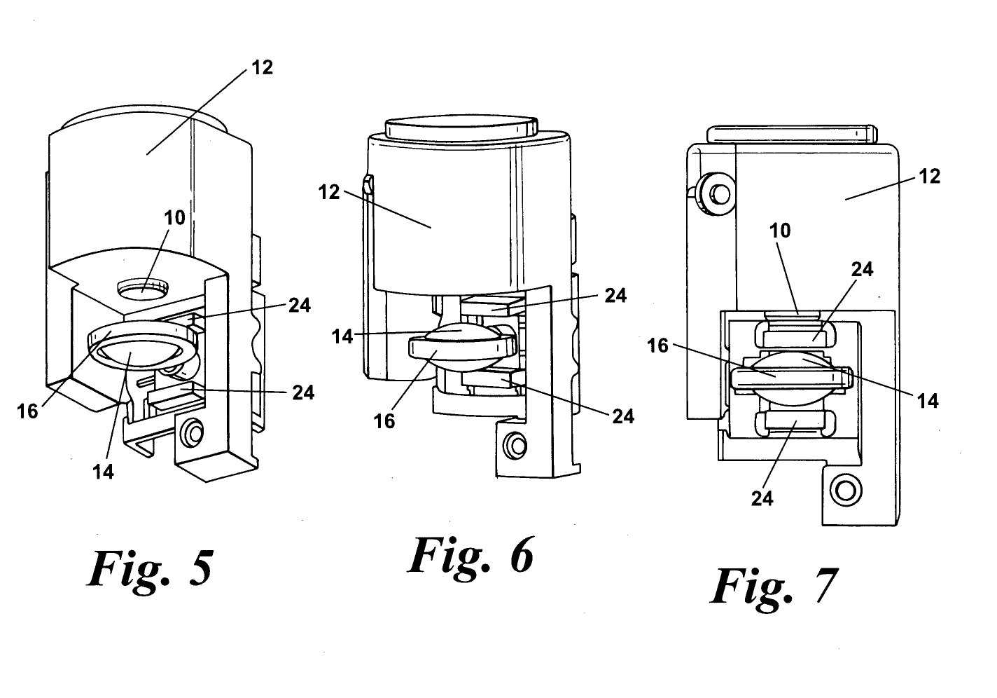

FIGS. 4 to 7 are further views of the assembly of FIG. 3;

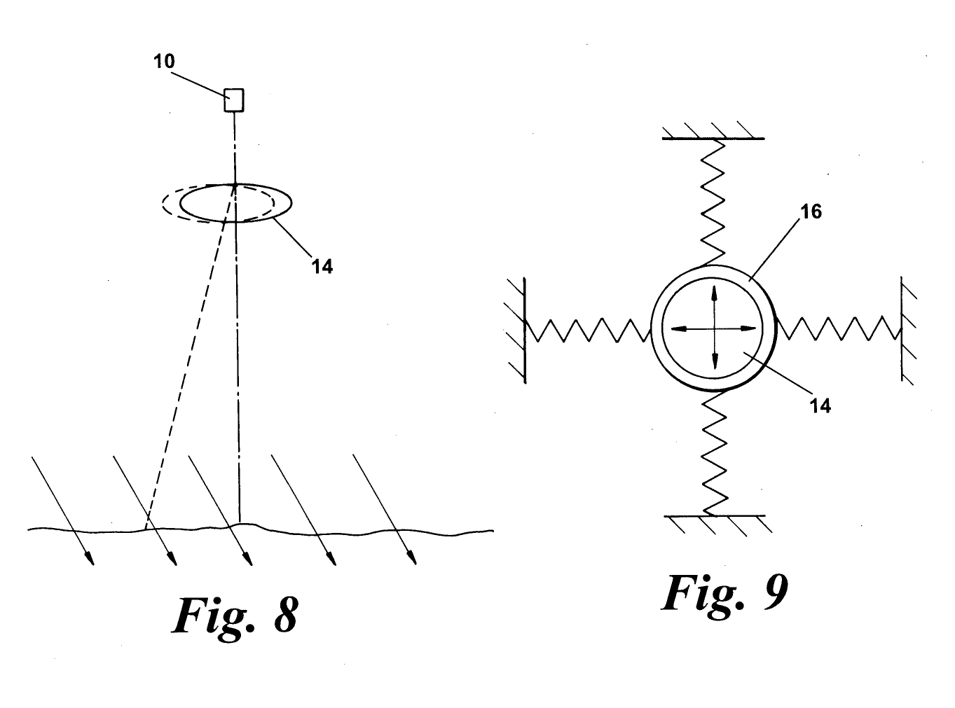

FIG. 8 is a schematic view of the optical arrangement showing movement of the lens to deflect the laser beam, and

FIG. 9 is a schematic view showing the mounting of the lens.

FIG. 1 depicts a portable device of the invention, shown generally at 1, which comprises a hand-held portion 2 which contains a laser (not shown) and associated optics (not shown) suitable for effecting hair removal by destroying hair follicles. The device 1 further comprises a main unit 3 which includes a recess shaped to receive the hand-held unit 2 when it is not in use. The main unit 3 further comprises a plurality of control buttons 4 for controlling the function of the device 1, such as selecting a desired treatment programme. The hand-held portion 2 is connected to the main unit 3 by way of a cord 5 which carries electrical power and control signals to the hand-held portion 2.

The internal arrangement of the portable device 1 is shown in more detail in FIG. 2. The main unit 3 contains a portable and rechargeable battery 6 which is used to power the device 1. The battery 6 powers the laser source 7, the power being transmitted through the cord 5 by an appropriate electrical connection. The battery 6 also powers the ancillary functions of the device 1, principally by supplying appropriate power to a controller 8. The controller 8 controls the operation of the laser 7 and also controls the operation of movable optic unit 9. The operation of the laser and movable optics will be described in more detail below with reference to FIGS. 3 to 9. Control signals from the controller 8 are routed to the laser 7 and movable optics unit 9 by appropriate electrical connections through the cord 5. The battery 6 is rechargeable, and appropriate recharging means 11 are provided which enable connections to be made (for example via a suitable input socket 11a) to a mains power supply for the purposes of recharging the battery 6. In this way, a device is provided which is truly portable, is not bulky in comparison to many prior art devices, has relatively low power consumption, and is easily utilised by an end user for effective self-treatment, even in respect of areas of the user's body which are not easily reached using relatively bulky prior art devices.

The preferred embodiment illustrated in the Figures discloses an apparatus in which a single laser beam is moved across the surface of the skin to be treated so as to sequentially target and destroy hair follicles. The movement of the laser beam will now be described in more detail. This aspect of the apparatus consists of a laser emitter, a focussing element (here a lens) which ensures that the beam delivers targeted energy to the target site, and a drive. In the various embodiments, the beam may be continuous so that it ‘sweeps’ the skin surface or it may be pulsed so that it moves in discrete steps. The movement of the beam may be controlled by one or more of the following: movement of a lens (as in the illustrated embodiment to be described below); movement of a mirror; and movement of the laser emitter. The movement may be achieved by means of an induction motor (as in the illustrated embodiment); a piezo-electric means, an electric motor mechanism with a transmission drive; a mechanical drive or any combination of the above.

In the embodiment described below, a laser emitter is used which passes the laser beam through a moveable lens. As the beam passes through the lens, it is subject to differing optical properties which result in the exiting laser beam passing through a different angle depending on which part of the lens it entered. Although the use of mirrors is not excluded, the use of a lens is preferred because reflective mirrors are generally expensive to manufacture; they are highly subject to optical degradation due to shock, moisture, heat and debris within the device, and moreover the commercial implementation requires use of a lens to focus the beam and so an existing component can be used thereby reducing the number of additional components that might otherwise compromise the reliability of the device.

Referring now to the illustrated embodiment shown in FIGS. 3 to 9, the device consists of a laser emitter 10, mounted in a suitable heat sink block 12, and a focusing lens 14 housed within a moveable support ring 16. The support ring is connected via a stub 18 to a printed circuit board (PCB) 20. The PCB is resiliently mounted for movement in two perpendicular directions in the plane of the lens by means of suitable flexural mountings (see FIG. 4). The PCB has two drive coils, an X drive coil and a Y drive coil (neither shown). The PCB 20 is supported between two permanent magnets 24. The X and Y coils on the PCB 20 and the permanent magnets 24 therefore operate similarly to the voice coils in a loudspeaker. The X direction coil moves the lens from left to right when viewed as in FIG. 7 and the Y direction coil moves the lens in and out as seen in that view. The applied voltage (negative or positive) to the coils determine the direction and amount of movement of the lens in either the X and Y direction. The voltages to the coils are controlled by a scan controller 26 shown schematically in FIG. 3.

In use, the controller 26 passes current through the X plane coil in PCB 20 so as to cause the lens to move in the X direction and this has the effect of causing the beam to track along the X plane of the skin as shown schematically in FIG. 8. When the beam is at a predetermined limit, the Y plane coil is also energised causing the beam to move in the Y direction. The X plane coil may then be de-energised causing the beam to retraces its movement, this time at a different Y position. Thus with coordinated control, the laser beam can be tracked across the skin in both the X and Y planes to scan a shaped treatment area.

In a preferred embodiment, the laser moves in steps equivalent to the diameter of the laser beam or scan spot, so as to uniformly treat an area of skin. Once the beam reaches the furthest extent of the X direction, the Y coil is energised and the beam moved by one laser diameter in the Y direction so as to provide a stepped form of raster scan. Scan movements may be pre-programmed or may be as a result of a sensory feedback from the skin surface indicating that the treatment in that area is complete. This could be by means of a suitable detector (such as an IR detector) detecting the temperature and determining that treatment in that area is complete.

Where the controller applies a stepped scan pattern, the controller may adjust the magnitude of the steps, decreasing with increasing angle of incidence, to take into account the divergence of the laser beam as the angle to the skin's normal becomes greater; it will be noted that the beam tends to diverge as this angle increases.

In another scan pattern, the controller moves the laser to scan it rapidly between two adjacent target sites. In this manner, the target sites experience a series of repeated exposures, somewhat similar to a fixed pulsed beam. By adjusting the duration and delay of the exposures the target site may receive sufficient repeated applications of energy to cause the hair to progressively heat up and prevent growth, whilst the temperature of the skin does not appreciably rise, because of the differing heating times and heating absorption/emission characteristics of the hairs and the surrounding tissue. Due to the relative melanin contents, hair absorbs laser energy more rapidly than the surrounding skin tissue and thus heats quicker. Furthermore, due to the geometry and anatomy of the hair compared to the surrounding tissue it loses heat slower. Thus by repeatedly subjecting the target area to discrete exposures to radiation it is possible to heat the hair follicle whilst keeping the surrounding skin at a nominal temperature. In the above embodiment, the effect similar to a pulse beam with radiation, is actually achieved by rapidly scanning between two adjacent target sites whilst the laser is continually powered in the avoidance of rapidly switching the laser on and off, thereby prolonging its operational life.

Various modifications to the device shown in the Figures are possible. For example, the portable battery might be disposed in the hand-held portion, or more than one portable battery might be provided. In the latter instance, two portable batteries might be provided, and one battery might be disposed in the main unit with the other battery being disposed in the hand-held portion. In embodiments in which more than one portable battery is provided, one battery might be used to power the laser, with another battery being used to power ancillary functions. In principle, the laser might be powered by more than one portable battery. The device may be configured so that the portable battery or batteries are automatically recharged when the hand-held portion is received by the main unit. In embodiments in which the laser is powered by a portable battery which is disposed in the hand-held portion, it is in principle possible to dispose with the cord. Control signals might be sent to the hand-held portion from the main unit by telemetry, or alternatively the control functions could be provided on the hand-held unit. It may be possible to provide a one-piece hand-held device, in which all of the device functionality is carried in or on a single hand-held portion.

1. A portable hair treatment device for the treatment of the human or animal skin by laser radiation, the portable device including:
a laser radiation source for emitting a laser radiation beam; and
a portable power source providing an electrical output for powering the laser radiation source.

2. A portable hair treatment device according to claim 1 in which the portable power source is a battery.

3. A portable hair treatment device according to claim 2 in which the portable power source is a rechargeable battery, and the portable device further includes recharging means for enabling the rechargeable battery to be recharged.

4. A portable hair treatment device according to claim 1 in which the portable power source is a fuel cell.

5. A portable hair treatment device according to claim 1 further including an ancillary portable power source for powering ancillary functions of the portable device.

6. A portable hair treatment device according to claim 3 including a hand-held portion which houses the laser radiation source and a receiving unit for receiving the hand-held portion when it is not in use.

7. A portable hair treatment device according to claim 6 in which the portable power source is located in the receiving unit.

8. A portable hair treatment device according to claim 6 in which the portable power source is located in the hand-held portion.

9. A portable hair treatment device according to claim 6 in which the portable power source is a rechargeable battery, and the recharging means is configured so that recharging commences when the receiving unit receives the hand-held portion.

10. A portable hair treatment device according to claim 1 having a single laser radiation source.

11. A device according to claim 1, wherein said treatment comprises cosmetic treatment.

12. A portable hair treatment device according to claim 1, wherein said treatment comprises therapeutic treatment.

13. A portable hair treatment device according to claim 11, wherein said treatment is effective for the removal of hair.

14. A portable hair treatment device according to claim 11, wherein said treatment is effective for the removal of tattoos or other pigmentation.

15. A portable hair treatment device according to claim 11, wherein said treatment is effective for treatment of blood vessels.

16. A portable hair treatment device according to claim 11, wherein said treatment is effective for the treatment of cellulite.

17. A portable hair treatment device according to claim 1, wherein said radiation source comprises a diode laser.

18. A portable hair treatment device according to claim 1, wherein said laser radiation source provides a laser beam having a wavelength of between 750 nm and 850 nm.

19. A portable hair treatment device according to claim 1 further including movement means for moving said laser radiation beam in a predetermined pattern across the skin.

20. A portable hair treatment device according to claim 19, wherein said movement means comprises an optical element mounted for movement and drive means for moving said optical element to deflect the beam axis.

21. A portable hair treatment device according to claim 20 in which the optical element is a lens.

22. A portable hair treatment device according to claim 19 wherein said movement means comprises said laser radiation source mounted for movement and drive means for moving said laser radiation source.

23. A portable hair treatment device according to claim 20, further including control means operable to control said drive means to deflect said beam.

24. A portable hair treatment device according to claim 19, in which the portable power source or an ancillary portable power source provides an electrical output for operating the movement means.

25. A portable skin treatment device for the treatment of the human or animal skin by laser radiation, the portable device including:
a laser radiation source for emitting a laser radiation beam, and
a portable power source providing an electrical output for powering the laser radiation source.
